(19) United States
(12) Patent Application Publication
(10) Pub. No.: US 2024/0000691 A1
(43) Pub. Date: Jan. 4, 2024

(54) Original Healing Gel

(71) Applicant: Danielle O'Neal, Columbia, SC (US)

(72) Inventor: Danielle O'Neal, Columbia, SC (US)

(21) Appl. No.: 17/852,555

(22) Filed: Jun. 29, 2022

(51) Int. Cl. A61K 8/73; A61K 8/92; A61K 8/25; A61K 8/34; A61Q 19/00

(52) U.S. Cl. CPC A61K 8/735; A61K 8/922; A61K 8/927; A61K 8/25; A61K 8/345; A61Q 19/007; A61K 2800/524

(57) ABSTRACT

The Healing Gel is a thick lotion for moisturizing dry skin comprised of mainly natural ingredients. The lotion is comprised of avocado oil, glycerin, polyglycerol-3 beeswax (available commercially as Cera Bellina Wax™), sodium hyaluronate dispersed in hydrogenated castor oil and castor seed oil (available commercially as Oleo HA™), a preservative (preferably a Geogard® Preservative), Mica, and fragrance.

### CROSS REFERENCE TO RELATED APPLICATIONS

None

### STATEMENT REGARDING FEDERALLY SPONSORED RESEARCH OR DEVELOPMENT

This invention was not made by an agency of the United States Government nor under a contract with an agency of the United States Government.

### THE NAME OF THE PARTIES TO JOINT RESEARCH AGREEMENT

Not Applicable.

### INCORPORATION BY REFERENCE OF MATERIAL SUBMITTED ON A COMPACT DISC OR AS A TEXT FILE VIA THE OFFICE ELECTRONIC FILING SYSTEM (EFS-WEB)

Not Applicable.

### STATEMENT REGARDING PRIOR DISCLOSURES BY THE INVENTOR OR A JOINT INVENTOR

Not Applicable

### BACKGROUND OF THE INVENTION

### Field of the Invention

Dry skin, also known as xerosis or xeroderma, has many causes, including cold or dry weather, overexposure to the sun, usage of harsh soaps, excessive bathing especially with hot water, and aging. Generally dry skin can be treated with moisturizers and other lotions. (www.mayoclinic.orddiseases-conditions/dry-skin/sysmptoms-causes/syc-20353885, accessed Jun. 21, 2022)

Many lotions and moisturizers contain a variety of chemicals which can cause problems for people with sensitive skin. “The Prevalence of Sensitive Skin” stated that 60-70% of women and of men report having some degree of sensitive skin. (www.ncbi/nlm.nih.gov/pmc/articles/PMC6533878/, accessed Jun. 21, 2022)

The present invention is a water free, natural skin lotion for the moisturization and treatment of dry skin. It contains no harsh chemicals which could irritate sensitive skin.

### Description of Related Art

A number of US patents for skin lotions utilize one or more of the ingredients of the present invention. However, the present invention is unique in the relative concentrations of the ingredients utilized. In particular, the percentage of avocado oil (approximately 88%) is the present invention is much greater than the prior art. In most instances where avocado oil is used, the concentration is less than 10%. The highest concentration of avocado oil identified was approximately 20% in U.S. Pat. No. 11,109,607 (Hall) and U.S. Pat. No. 9,724,290 (Fairley).

### BRIEF SUMMARY OF THE INVENTION

The present invention is a thick lotion for moisturizing dry skin comprised of mainly natural ingredients. The lotion is comprised of avocado oil, glycerin, Cera Bellina Wax, Oleo HA, Geogard Preservative, Mica, and fragrance. A method for making the lotion is also disclosed.

### BRIEF DESCRIPTION OF THE DRAWING

FIG. 1 is a flow chart showing the steps to prepare 1 kg of the product.

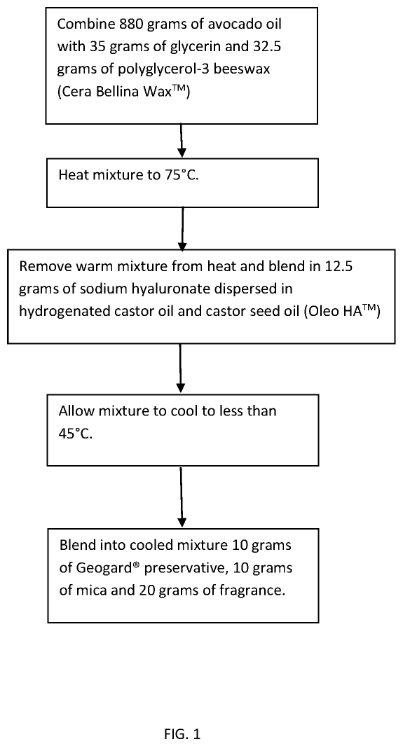

### DETAILED DESCRIPTION OF THE INVENTION

The preferred embodiment of the Original Healing Gel is comprised of 88% avocado oil, 3.5% glycerin, 3.25% Cera Bellina Wax, 1.25% Oleo HA, 1% Geogard Preservative, 1% Mica, and 2% fragrance. The healing gel is prepared by mixing the avocado oil, glycerin, and Cera Bellina Wax, then heating the mixture to 75° C. The warm mixture is removed from the heat source and Oleo HA is blended in and the resulting mixture allowed to cool to less than 45° C. Geogard Preservative, mica and fragrance are then added to the cooled mixture to create the final product.

The composition disclosed in paragraph 013 is the preferred embodiment of the product. However, the compositional percentages can vary as shown in Table 1.

TABLE 1
Composition of Original Healing Gel

| Component | Preferred Embodiment % | % Range |
|---|---|---|
| Avocado Oil | 88 | 80-96 |
| Glycerin | 3.5 | 3-4 |
| Cera Bellina Wax | 3.25 | 2.75-3.75 |
| Oleo HA | 1.25 | 1-1.5 |
| Geogard Preservative | 1 | 0.5-1.5 |
| Mica | 1 | 0.5-1.5 |
| Fragrance | 2 | 1-3 |

Avocado oil is the main ingredient of the Original Healing Gel. The concentration of avocado oil in the gel is one of the distinctive features of the invention. The quantity of avocado oil in the healing gel is at least 4 times the amount of avocado oil utilized in prior art lotions.

The beta carotene, protein, lecithin, fatty acids, and vitamins A, D, and E present in avocado oil helps moisturize the skin and protect it from damaging UV rays. (www.healthline.com/health/beauty-skin-care/avocado-oil-for-skin#reasearch, accessed Jun. 28, 2022). An animal study in 2013 found that the linoleic acid, oleic acid and other monosaturated fatty acids in avocado oil can speed up wound healing. (www.ncbi.nlm.nih.gov/pmc/articles/PMC3614059/, accessed Jun. 28, 2022) The vitamins, minerals and essential fatty acids contained in avocado oil aid in collagen production, skin nourishment, antioxidant effects, and anti-inflammatory effects all of which promote healthy skin.

The Healing Gel is a water free emulsion in which glycerin is used to replace the water component of a typical emulsion. Glycerin, also known as glycerol, is a natural compound derived from vegetable oils or animal fat. Glycerin is a humectant, which is a type of moisturizing agent that pulls water into the outer layer of your skin from deeper levels of your skin and the air. (www.healthline.com/health/glycerin-for-face, accessed Jun. 28, 2022)

Cera Bellina Wax is a hydrophilic derivative of natural beeswax in which the free fatty acids have been converted to polyglycerols esters. It is used in the present invention as an emulsifier, thickener, and a stabilizing agent to prevent the separation of the product components. Additionally, Cera Bellina Wax aids in imparting a smooth, satin-like feeling to the skin and is suitable for use for sensitive skin.

Oleo HA is a dispersion of sodium hyaluronate in hydrogenated castor oil and castor seed oil. When applied to the skin, sodium hyaluronate attracts moisture to skin cells and thus, increase skin hydration. Studies have also shown that sodium hyaluronate can also sooth skin inflammation and aid in the healing of wounds. (www.healthline.com/health/beauty-skin-care/sodium-hyaluronate, accessed Jun. 28, 2022)

Micas are a group of silicate minerals used in cosmetics to add shimmer to the product. It can also provide color to the product. A preservative is included in the formulation of the invention product to increase the shelf-life of the product. Fragrance in various scents is also utilized.

Suggested classifications for this invention include:
A61K 8/04: Cosmetics or similar toilet preparations, Dispersions, Emulsions
A61K 8/92: Cosmetics or similar toilet preparations, Oils, fats, or waxes
A61Q 19/00: Preparations for care of the skin
A61Q 19/007: Preparations for dry skin

The terminology used herein is for describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

The invention claimed is:

1. A lotion for the treatment of dry skin comprising 80 to 96% w/w avocado oil, 3 to 4% w/w glycerin, 2.75 to 3.75% w/w polyglycerol-3 beeswax, 1 to 1.5% w/w sodium hyaluronate dispersed in castor oil and castor seed oil, 0.5 to 1.5% w/w preservative, 0.5-1.5% w/w mica, and 1 to 3% w/w fragrance.

2. The lotion of claim 1, where said lotion comprises 88% w/w avocado oil, 3.5% w/w glycerin, 3.25% w/w polyglycerol-3 beeswax, 1.25% w/w sodium hyaluronate dispersed in castor oil and castor seed oil, 1% w/w preservative, 1% w/w mica, and 2% w/w fragrance.

3. The lotion of claim 1 prepared by mixing avocado oil, glycerin and polyglycerol-3 beeswax, and heating said mixture to 75° C., then removing said mixture away from the heating source and adding sodium hyaluronate dispersed in castor oil and castor seed oil to said mixture, then cooling to less than 45° C. and then adding preservative, mica, and fragrance.
